(19) United States
(12) Patent Application Publication
(10) Pub. No.: US 2013/0296233 A1
(43) Pub. Date: Nov. 7, 2013

(54) Dietary Supplement and Methods of Use Thereof

(71) Applicant: Elizabeth C. Theil, Oakland, CA (US)

(72) Inventor: Elizabeth C. Theil, Oakland, CA (US)

(73) Assignee: CHILDREN'S HOSPITAL & RESEARCH CENTER AT OAKLAND, Oakland, CA (US)

(21) Appl. No.: 13/814,897

(22) PCT Filed: Sep. 7, 2011
(86) PCT No.: PCT/US11/50650

§ 371 Date: Apr. 10, 2013

Related U.S. Application Data
(60) Provisional application No. 61/380,962, filed on Sep. 8, 2010.

(51) Int. Cl. A61K 38/16; A23L 1/30

(52) U.S. Cl. CPC A61K 38/16; A23L 1/3002
USPC 514/5.5

(57) ABSTRACT

The present disclosure provides a dietary supplement that includes plant ferritin; and methods of use of such a supplement.

### CROSS-REFERENCE

This application claims the benefit of U.S. Provisional Patent Application No. 61/380,962, filed Sep. 8, 2010, which application is incorporated herein by reference in its entirety.

### STATEMENT REGARDING FEDERALLY SPONSORED RESEARCH

This invention was made with government support under Grant Nos. HL562169 and DK20251 awarded by National Institutes of Health. The government has certain rights in the invention.

### BACKGROUND

In the human body, iron is essential for the implementation and maintenance of many vital cellular functions and biosynthetic processes, including oxygen transport capabilities, aerobic cellular activity, intracellular electron transport, and integral enzymatic reactions within body tissue. Iron deficiency is the most common nutritional deficiency worldwide, affecting 30 million people in both developed and developing countries. Iron deficiency diminishes growth and learning in children.

Ferritin is a large multisubunit protein that stores and concentrates iron a protein-coated iron mineral in plants, animals, and bacteria. Ferritin is a natural, slow-release form of biologically available iron with up to 4,500 iron atoms as Fe₂O₃.H₂O inside a self-assembling protein cage. Ferritin concentrates iron in cells 100 billion times above the solubility of ferric ion in a nontoxic, accessible form. The subunits, four α-helix bundles, contain a catalytic center that converts two Fe(II) atoms to an Fe(III)-oxo bridged dimer intermediate in mineralization. The two classes of ferritins are: i) maxi-ferritins, 24-polypeptide, 4-bundle subunit assemblies found in animals, plants, and bacteria; and ii) mini-ferritins (also called Dsp proteins), 12-polypeptide, 4-bundle subunit assemblies in archaea and bacteria.

### Literature

Beard et al. (1996) J. Nutr. 126:154; Theil (2003) J. Nutr. 133:1549S; Murray-Kolb et al. (2002) J. Nutr. 132:957; Murray-Kolb et al. (2003) Am. J. Clin. Nutr. 77:180; Davila-Hicks et al. (2004) Am. J. Clin. Nutr. 80:936; Liu et al. (2006) Biol. Res. 39:167; WO 1999/002687; US 2008/0248132; Hasan et al. (2008) J. Biol. Chem. 283:31394; Lonnerdal et al. (2006) Am. J. Clin. Nutr. 83:103; U.S. Pat. No. 7,659,074; U.S. Pat. No. 7,754,702; U.S. Pat. No. 7,160,855; Barnes et al. (2002) Proc. Natl. Acad. Sci. USA 99:5195-5200; Masuda et al. (2001) J. Biol. Chem. 276:19575.

### SUMMARY OF THE INVENTION

The present disclosure provides a dietary supplement that includes plant ferritin; and methods of use of such a supplement.

### DEFINITIONS

As used herein, “ferritin” refers to a ferritin protein nanocage comprising mineralized iron, and including H and/or L ferritin polypeptide subunits.

As used herein, “pharmaceutically acceptable carrier” includes any material which, when combined with an active ingredient of a composition, allows the ingredient to retain biological activity and without causing disruptive reactions with the subject's immune system. Examples include, but are not limited to, any of the standard pharmaceutical carriers such as a phosphate buffered saline solution, water, emulsions such as oil/water emulsion, and various types of wetting agents. Exemplary diluents for aerosol or parenteral administration are phosphate buffered saline or normal (0.9%) saline. Compositions comprising such carriers are formulated by well known conventional methods (see, for example, Remington's Pharmaceutical Sciences, Chapter 43, 14th Ed. or latest edition, Mack Publishing Co., Easton Pa. 18042, USA; A. Gennaro (2000) “Remington: The Science and Practice of Pharmacy”, 20th edition, Lippincott, Williams, & Wilkins; Pharmaceutical Dosage Forms and Drug Delivery Systems (1999) H. C. Ansel et al., eds 7^{th} ed., Lippincott, Williams, & Wilkins; and Handbook of Pharmaceutical Excipients (2000) A. H. Kibbe et al., eds., 3^{rd} ed. Amer. Pharmaceutical Assoc.

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment,” as used herein, covers any treatment of a disease in a mammal, particularly in a human, and can include: (a) preventing the disease or a symptom of a disease from occurring in a subject which may be predisposed to the disease but has not yet been diagnosed as having it (e.g., including diseases that may be associated with or caused by a primary disease); (b) inhibiting the disease or condition, i.e., arresting its development; and (c) relieving the disease, i.e., causing regression of the disease. The term “ameliorating” or “ameliorate” refers to any indicia of success in the treatment of a pathology or condition, including any objective or subjective parameter such as abatement, remission or diminishing of symptoms or an improvement in a patient's physical or mental well-being. Amelioration of symptoms can be based on objective or subjective parameters; including the results of a physical examination.

The terms “individual,” “host,” “subject,” and “patient” are used interchangeably herein, and generally refer to a mammal, including, but not limited to, humans; non-human primates such as simians; equines (e.g., horses); canines (e.g., dogs); felines; rodents (e.g., mice; rats); various domesticated livestock (e.g., ungulates, such as swine, pigs, goats, sheep, and the like); as well as domesticated mammalian pets and mammals maintained in zoos.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a plant ferritin” includes a plurality of such supplements and reference to “the dietary supplement” includes reference to one or more dietary supplements and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

The present disclosure provides a dietary supplement that includes plant ferritin; and methods of use of the dietary supplement to treat iron deficiency.

### Dietary Supplement

The present disclosure provides a dietary supplement, which can be in the form of a pharmaceutical formulation, or in the form of a food product, where a subject dietary supplement comprises plant ferritin in an amount of from about 10% to about 50% by weight of the total protein. The plant ferritin is prepared from a legume, or a fraction or subportion of a legume. The whole legume can be used; or a fraction or subportion, such as the hull of a legume, can be used as the source of the plant ferritin.

A subject dietary supplement is useful for treating various disorders and conditions, as described in more detail below. A subject dietary supplement is formulated for oral administration, e.g., for oral consumption.

Suitable legume sources of ferritin include, but are not limited to, soybeans; beans, yellow peas, green peas, lentils, chickpeas, peanuts, trefoil, pinto beans, Great Northern beans, navy beans, red beans, black beans, dark or light red kidney beans, fava beans, green baby lima beans, pink beans, mayocoba beans, small red beans, black-eyed peas, garbanzo beans (also called “chick peas”), cranberry beans, white beans, rice beans, butter beans, and combinations of any of the foregoing. See, e.g., Theil (2004) Annu. Rev. Nutr. 24:327; Sczekan et al. (1987) J. Biol. Chem. 262:13780; Laulhere et al. (1988) J. Biol. Chem. 263:10289; Wardrop et al. (1999) Biochem. J. 337:523; Masuda et al. (2001) J. Biol. Chem. 276:19575. The legume can be any of a variety of species, including, e.g., a Phaseolus species (e.g,. Phaseolus vulgaris), a Pisum species (e.g., Pisum sativum), a Lens species (e.g., Lens vulgaris, Lens culinaris), a Cicera species (e.g., Cicera arietenum), a Vigna species (e.g., Vigna unguiculata), a Glycine species (e.g., Glycine max), and combinations of any thereof.

The source of the ferritin can include the whole plant, or any ferritin-rich portion of a plant, e.g., seed, stem, fruit, leaf, root (e.g., nodulating root), flower, stem, etc. In some cases, the source of the ferritin is one or more of a seed, a nodulating root, and a leaf. Where the source of the ferritin is a seed or a bean, the ferritin can be obtained from the whole seed or bean, or a part of a seed or bean, e.g., the hull. The source of the ferritin can be a waste stream from processing of soy or other bean. For example, the source of the ferritin can be a waste stream (e.g., okara) from production of tofu from soybeans. The source of the ferritin can be an extract of the whole seed or seed part (e.g., hull; cotyledon; hypocotyl).

A subject dietary supplement includes ferritin in an amount of from about 10% to about 50% by weight of the total protein in the supplement. For example, subject dietary supplement includes ferritin in an amount of from about 10% to about 15%, from about 15% to about 20%, from about 20% to about 25%, from about 25% to about 30%, from about 30% to about 35%, from about 35% to about 40%, from about 40% to about 45%, or from about 45% to about 50% by weight of the total protein in the supplement.

### Pharmaceutical Compositions

In some embodiments, a subject dietary supplement is in the form of a pharmaceutical composition. A subject pharmaceutical composition comprises a subject dietary supplement; and a pharmaceutically acceptable carrier.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

For oral preparations, a subject dietary supplement can be formulated with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

### Food Products

In some embodiments, a subject dietary supplement is in the form of a food product. A subject food product comprises a subject dietary supplement; and can further include one or more food-grade components.

In some embodiments, the plant ferritin is formulated with one or more food-grade components, e.g., a dosage form is a nutraceutical or a food product. The term “nutraceutical formulation” refers to a food or part of a food that offers medical and/or health benefits including prevention or treatment of disease. Nutraceutical products range from isolated nutrients, dietary supplements and diets, to genetically engineered designer foods, functional foods, herbal products and processed foods such as cereal, soup and beverages. The term “functional foods,” refers to foods that include “any modified food or food ingredients that may provide a health benefit beyond the traditional nutrients it contains.” Thus, by definition, pharmaceutical compositions comprising a plant ferritin include nutraceuticals. Also by definition, pharmaceutical compositions comprising a therapeutic nucleic acid include compositions comprising a plant ferritin and a food-grade component. A plant ferritin may be added to food products to provide a health benefit.

Nutraceutical formulations of interest include foods for veterinary or human use, including food bars (e.g. cereal bars, breakfast bars, energy bars, nutritional bars); chewing gums; drinks; fortified drinks; drink supplements (e.g., powders to be added to a drink); tablets; lozenges; candies; and the like. These foods are enhanced by the inclusion of plant ferritin. For example, in the treatment of iron deficiency, the normal diet of a patient may be supplemented by a subject plant ferritin nutraceutical formulation taken on a regular basis, e.g., at meal times, before meals, or after meals.

The present disclosure provides compositions (e.g., nutraceutical compositions) comprising plant ferritin and a food-grade pharmaceutically acceptable excipient. In many embodiments, plant ferritin nutraceutical compositions include one or more components found in food products. Thus, the instant disclosure provides a food composition and products comprising plant ferritin and a food component. Suitable components include, but are not limited to, mono- and disaccharides; carbohydrates; proteins; amino acids; fatty acids; lipids; stabilizers; preservatives; flavoring agents; coloring agents; sweeteners; antioxidants, chelators, and carriers; texturants; nutrients; pH adjusters; emulsifiers; stabilizers; milk base solids; edible fibers; and the like. The food component can be isolated from a natural source, or can be synthesized. All components are food-grade components fit for human consumption.

Examples of suitable monosaccharides include sorbitol, mannitol, erythrose, threose, ribose, arabinose, xylose, ribulose, glucose, galactose, mannose, fructose, and sorbose. Non-limiting examples of suitable disaccharides include sucrose, maltose, lactitol, maltitol, maltulose, and lactose.

Suitable carbohydrates include oligosaccharides, polysaccharides, and/or carbohydrate derivatives. As used herein, the term “oligosaccharide” refers to a digestible linear molecule having from 3 to 9 monosaccharide units, wherein the units are covalently connected via glycosidic bonds. As used herein, the term “polysaccharide” refers to a digestible (i.e., capable of metabolism by the human body) macromolecule having greater than 9 monosaccharide units, wherein the units are covalently connected via glycosidic bonds. The polysaccharides may be linear chains or branched. Carbohydrate derivatives, such as a polyhydric alcohol (e.g., glycerol), may also be utilized as a complex carbohydrate herein. As used herein, the term “digestible” in the context of carbohydrates refers to carbohydrate that are capable of metabolism by enzymes produced by the human body. Examples of polysaccharides non-digestible carbohydrates are resistant starches (e.g., raw corn starches) and retrograded amyloses (e.g., high amylose corn starches). Non-limiting examples carbohydrates include raffinoses, stachyoses, maltotrioses, maltotetraoses, glycogens, amyloses, amylopectins, polydextroses, and maltodextrins.

Suitable fats include, but are not limited to, triglycerides, including short-chain (C₂-C₄) and long-chain triglycerides (C₁₆-C₂₂).

Suitable texturants (also referred to as soluble fibers) include, but are not limited to, pectin (high ester, low ester); carrageenan; alginate (e.g., alginic acid, sodium alginate, potassium alginate, calcium alginate); guar gum; locust bean gum; psyllium; xanthan gum; gum arabic; fructo-oligosaccharides; inulin; agar; and functional blends of two or more of the foregoing.

Suitable emulsifiers include, but are not limited to, propylene glycol monostearate (PGMS), sodium stearoyl lactylate (SSL), calcium stearoyl lactylate (CSL), monoglycerides, diglycerides, monodiglycerides, polyglycerol esters, lactic acid esters, polysorbate, sucrose esters, diacetyl tartaric acid esters of mono-diglycerides (DATEM), citric acid esters of monoglycerides (CITREM) and combinations thereof. Additional suitable emulsifiers include DIMODAN distilled monoglycerides, including DIMODAN™ B 727 and DIMODAN™ PV, GRINDSTED™ CITREM, GRINDSTED™ GA, GRINDSTED™ PS such as GRINDSTED™ PS 100, GRINDSTED™ PS 200, GRINDSTED™ PS 300, GRINDSTED™ PS 400; RYLO™ (manufactured and distributed by DANISCO CULTOR), including RYLO™ AC, RYLO™ CI, RYLO™ LA, RYLO™ MD, RYLO™ MG, RYLO™ PG, RYLO™ PR, RYLO™ SL, RYLO™ SO, RYLO™ TG; and combinations thereof.

Edible fibers include polysaccharides, oligosaccharides, lignin and associated plant substances. Suitable edible fibers include, but are not limited to, sugar beet fiber, apple fiber, pea fiber, wheat fiber, oat fiber, barley fiber, rye fiber, rice fiber, potato fiber, tomato fiber, other plant non-starch polysaccharide fiber, and combinations thereof.

Suitable flavoring agents include natural and synthetic flavors, “brown flavorings” (e.g., coffee, tea); dairy flavorings; fruit flavors; vanilla flavoring; essences; extracts; oleoresins; juice and drink concentrates; flavor building blocks (e.g., delta lactones, ketones); and the like; and combinations of such flavors. Examples of botanic flavors include, for example, tea (e.g., preferably black and green tea), aloe vera, guarana, ginseng, ginkgo, hawthorn, hibiscus, rose hips, chamomile, peppermint, fennel, ginger, licorice, lotus seed, schizandra, saw palmetto, sarsaparilla, safflower, St. John's Wort, curcuma, cardamom, nutmeg, cassia bark, buchu, cinnamon, jasmine, haw, chrysanthemum, water chestnut, sugar cane, lychee, bamboo shoots, vanilla, coffee, and the like.

Suitable sweeteners include, but are not limited to, alitame; dextrose; fructose; lactilol; polydextrose; xylitol; xylose; aspartame, saccharine, cyclamates, acesulfame K, L-aspartyl-L-phenylalanine lower alkyl ester sweeteners, L-aspartyl-D-alanine amides; L-aspartyl-D-serine amides; L-aspartyl-hydroxymethyl alkane amide sweeteners; L-aspartyl-1-hydroxyethylalkane amide sweeteners; and the like.

Suitable anti-oxidants include, but are not limited to, tocopherols (natural, synthetic); ascorbyl palmitate; gallates; butylated hydroxyanisole (BHA); butylated hydroxytoluene (BHT); tert-butyl hydroquinone (TBHQ); and the like.

Suitable nutrients include vitamins and minerals, including, but not limited to, niacin, thiamin, folic acid, pantothenic acid, biotin, vitamin A, vitamin C, vitamin B₂, vitamin B₃, vitamin B₆, vitamin B₁₂, vitamin D, vitamin E, vitamin K, iron, zinc, copper, calcium, phosphorous, iodine, chromium, molybdenum, and fluoride.

Suitable coloring agents include, but are not limited to, FD&C dyes (e.g., yellow #5, blue #2,red #40), FD&C lakes; Riboflavin; β-carotene; natural coloring agents, including, for example, fruit, vegetable, and/or plant extracts such as grape, black currant, aronia, carrot, beetroot, red cabbage, and hibiscus.

Exemplary preservatives include sorbate, benzoate, and polyphosphate preservatives.

Suitable emulsifiers include, but are not limited to, diglycerides; monoglycerides; acetic acid esters of mono- and diglycerides; diacetyl tartaric acid esters of mono- and diglycerides; citric acid esters of mono- and diglycerides; lactic acid esters of mono- and diglycerides; fatty acids; polyglycerol esters of fatty acids; propylene glycol esters of fatty acids; sorbitan monostearates; sorbitan tristearates; sodium stearoyl lactylates; calcium stearoyl lactylates; and the like.

Suitable agents for pH adjustment include organic as well as inorganic edible acids. The acids can be present in their undissociated form or, alternatively, as their respective salts, for example, potassium or sodium hydrogen phosphate, potassium or sodium dihydrogen phosphate salts. Exemplary acids are edible organic acids which include citric acid, malic acid, fumaric acid, adipic acid, phosphoric acid, gluconic acid, tartaric acid, ascorbic acid, acetic acid, phosphoric acid and mixtures thereof.

In some embodiments, a subject food product/nutraceutical formulation does not include a substantial amount of heme iron, ferrous sulfate, ferrous gluconate, ferrous fumarate, or any other source of iron other than ferritin.

Plant ferritin is present in the food product/nutraceutical formulation in an amount of from about 5% to about 7%, from about 7% to about 10%, from about 10% to about 15%, from about 15% to about 20%, from about 20% to about 25%, from about 25% to about 30%, from about 30% to about 35%, from about 35% to about 40%, from about 40% to about 45%, or from about 45% to about 50%, by weight.

Where the food product is a beverage, the food product generally contains, by volume, more than about 50% water, e.g., from about 50% to about 60%, from about 60% to about 95% water, e.g., from about 60% to about 70%, from about 70% to about 80%, from about 80% to about 90%, or from about 90% to about 95% water.

Where the food product is a solid or semi-solid food product, e.g., a bar, tablet, solid candy, lozenge, etc., the food product generally contains, by volume, less than about 15% water, e.g., from about 2% to about 5%, from about 5% to about 7%, from about 7% to about 10%, from about 10% to about 12%, or from about 12% to about 15% water.

In some embodiments, the food product is essentially dry, e.g., comprises less than about 5%, water.

Monosaccharides, disaccharides, and complex carbohydrates, if present, are generally present in an amount of from about 0.1% to about 15%, e.g., from about 0.1% to about 1%, from about 1% to about 5%, from about 5% to about 7%, from about 7% to about 10%, or from about 10% to about 15%, by weight each. Soluble fibers, edible fibers, and emulsifiers, if present, are generally present in an amount of from about 0.1% to about 15%, e.g., from about 0.1% to about 1%, from about 1% to about 5%, from about 5% to about 7%, from about 7% to about 10%, or from about 10% to about 15%, by weight each.

Other components discussed above, if present, are present in amounts ranging from about 0.001% to about 5% by weight of the composition.

### Food Formulations and Pharmaceutical Formulations

A subject formulation can be prepared in a variety of ways for consumption by an individual, and, as indicated above, can include one or more food components. Food formulations can be in a variety of forms, including powders; liquids; gels; semi-solid; and solid forms such as bars, tablets, capsules, candies, etc. Formulations of interest include foods for veterinary or human use, including food bars (e.g. cereal bars, breakfast bars, energy bars, nutritional bars); drinks; fortified drinks; carbonated beverages; drink supplements (e.g., powders to be added to a drink); powders to be mixed with food; tablets; lozenges; candy; candy-like formulations, e.g., chewable gel formulations, e.g., chewable gel candy in the shape of an animal; puddings; gels; mousses; a blended drink (e.g., a “smoothie”); and the like. Suitable food formulations also include those described in U.S. Pat. No. 7,067,150.

A food product can have final moisture content between about 0% and about 100%, e.g., from about 0% to about 1%, from about 1% to about 5%, from about 5% to about 10%, from about 10% to about 30%, from about 30% to about 50%, from about 50% to about 80%, or from about 80% to about 100%.

In some embodiments, a unit dosage form of a subject dietary supplement is a food bar, where a food bar unit dosage form can have a weight of from about 15 grams to about 50 grams, e.g., 15 g to 20 g, from 20 g to 25 g, from 25 g to 30 g, from 30 g to 35 g, from 35 g to 40 g, from 40 g to 45 g, or from 45 g to 50 g.

In some embodiments, a unit dosage form of a subject dietary supplement is a liquid, e.g., a beverage, where a beverage unit dosage form can have a volume of from 4 ounces to 16 ounces, e.g., from 4 oz to 6 oz, from 6 oz to 8 oz, from 8 oz to 10 oz, from 10 oz to 12 oz, from 12 oz to 14 oz, or from 14 oz to 16 oz.

In some embodiments, a unit dosage form of a subject dietary supplement is a semi-solid (e.g., having the consistency of a pudding, a yogurt, and the like), e.g., where a semi-solid unit dosage form can have a volume of from 4 ounces to 16 ounces, e.g., from 4 oz to 6 oz, from 6 oz to 8 oz, from 8 oz to 10 oz, from 10 oz to 12 oz, from 12 oz to 14 oz, or from 14 oz to 16 oz.

### Food Bars

In some embodiments, a subject food product is in the form of a bar, e.g., a food bar. A subject food bar includes plant ferritin, as described above, and can further include one or more additional food-grade components, e.g., palatability enhancers, and the like. In some embodiments, a subject food bar comprises, in addition to plant ferritin, a fruit puree, e.g., a puree of one or more of the following fruits: blueberry, raspberry, strawberry, pear, peach, apple, blackberry, mango, kiwi, etc. In some embodiments, a subject food bar comprises, in addition to plant ferritin, one or more different nuts and/or seeds, e.g., one or more of walnuts, hazelnuts, sunflower seeds, peanuts, almonds, pecans, sesame seeds, etc. In some embodiments, where a subject food bar comprises one or more nuts or seeds, the nuts or seeds are ground, pureed, or provided in the form of a paste or butter (e.g., peanut butter, almond butter, etc.). In some embodiments, where a subject food bar comprises one or more nuts or seeds, a nut or a seed that may induce an allergic response and/or anaphylaxis in an individual is not included. Thus, e.g., in some embodiments, peanuts are excluded. In some embodiments, a subject food bar comprises, in addition to plant ferritin, chocolate. In some embodiments, a subject food bar comprises, in addition to plant ferritin, chocolate and one or more nuts or seeds. In some embodiments, a subject food bar comprises, in addition to plant ferritin, one or more nuts or seeds, and a fruit puree. In some embodiments, a subject food bar comprises, in addition to plant ferritin, coffee, e.g., coffee powder, coffee beans, where the coffee is in some embodiments caffeinated and in other embodiments decaffeinated. In some embodiments, a subject food bar comprises, in addition to plant ferritin, citric acid.

### Packages

The present invention further provides a package comprising a subject formulation. In some embodiments, a subject package comprises a single dosage form of a subject formulation. In other embodiments, a subject package a subject package comprising multiple (e.g., 2, 3, 4, 5, 6, 7, 8, 9, 10, or more) dosage forms of a subject formulation.

As one non-limiting example, a subject food product can be packaged in such a way that multiple doses are contained in a single package, optionally where individual unit dosage forms are separated in individual compartments in a single package. The dosage forms can be in a variety of forms, e.g., tablets or lozenges that are palatable (e.g., flavored so as to be palatable, such as with fruit flavorings, sugars, and the like, as discussed above). Unit dosage forms include tablets, capsules, lozenges, candies, bars, a unit of powder (e.g., 1 tablespoon of a powder; a unit of a liquid, (e.g., a 1 tablespoon of a liquid), a unit of a semi-solid, etc.

A subject package in some embodiments will further include instructions for use, including e.g., dosage amounts and dosage frequencies. Instructions are in some embodiments printed directly on the package. In other embodiments, instructions are printed material provided as a package insert. Instructions can also be provided in other media, e.g., electronically in digital or analog form, e.g., on an audio cassette, an audio tape, a compact disc, a digital versatile disk, and the like.

### Treatment Methods

The present disclosure provides a method for treating iron deficiency, and/or a disorder associated with iron deficiency, the method comprising administering to an individual in need thereof (e.g., a human in need thereof) an effective amount of a subject dietary supplement. Target populations and individuals (e.g., individuals, and populations of individuals, in need of treatment with a subject dietary supplement) include, but are not limited to, children; adolescents; patients on kidney dialysis; women of child-bearing age; elderly individuals (e.g., individuals over the age of 65, e.g., individuals ranging in age from about 65 year to about 70 years, from about 70 years to about 80 years, from about 80 years to about 90 years, from about 90 years to about 100 years, and older than 100 years); pregnant women (including pregnant females of any age); individuals (and populations of individuals) who have limited access to and/or economic means to acquire, adequate nutrition (e.g., iron nutrition).

Individuals with limited access to and/or means for acquiring adequate nutrition include, e.g., individuals with limited access to and/or means for acquiring adequate nutrition due to socioeconomic and/or environmental deprivation, e.g., inner city minority children; individuals with limited access to and/or means for acquiring adequate nutrition due to conditions such as economic disruption resulting from war; individuals with limited access to and/or means for acquiring adequate nutrition (e.g., iron nutrition) due to occupation, e.g., military personnel in a combat zone; and the like.

A subject dietary supplement can be administered in one, or more than one, doses per day, e.g., a subject dietary supplement can be administered in one, two, three, or four doses in a single day. A subject dietary supplement can be administered at various frequencies, e.g., four times daily, three times daily, twice daily, once daily, every other day, three times per week, twice per week, or once per week. In a particular embodiment, a subject dietary supplement is administered once daily. In another particular embodiment, a subject dietary supplement is administered twice daily.

The amount that is considered an “effective amount” of a subject dietary supplement will vary, depending, e.g., on the nature or severity of the disorder or condition being treated, the age and/or physical condition of the individual being treated, and the like.

A subject dietary supplement can be administered in one, or more than one, doses per day, e.g., a subject dietary supplement can be administered in one, two, three, or four doses in a single day. A subject dietary supplement can be administered at various frequencies, e.g., four times daily, three times daily, twice daily, once daily, every other day, three times per week, twice per week, or once per week. In a particular embodiment, a subject dietary supplement is administered once daily. For example, in some embodiments, a unit dosage form of a subject dietary supplement is administered once daily. In another particular embodiment, a subject dietary supplement is administered twice daily. For example, in some embodiments, a unit dosage form of a subject dietary supplement is administered twice daily. As another example, in some embodiments, a unit dosage form of a subject dietary supplement is administered three times daily. As another example, in some embodiments, a unit dosage form of a subject dietary supplement is administered four times daily.

Whether an individual has received an effective amount of a subject dietary supplement can be readily determined by any standard method, e.g., hematocrit.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Example 1

### Preparation of Plant Ferritin

“Waste” processing streams from the preparation of tofu or bean protein proteins are typically high in iron, and are concentrated (water decreased) by evaporation, with or without low heat, before being used as a supplement and iron concentrate.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A dietary supplement,
wherein the supplement comprises plant ferritin in an amount of from about 10% to about 50% by weight of the total protein,
and wherein the supplement is prepared from at least the seeds and hull of a legume.

2. The supplement of claim 1, wherein the supplement comprises at least one food-grade carrier.

3. The supplement of claim 2, wherein the food-grade carrier is selected from the group consisting of olive oil, an emulsifier, a soluble fiber, a flavoring agent, a coloring agent, an edible fiber, and a sweetener.

4. The supplement of claim 3, wherein the soluble fiber is selected from the group consisting of pectin, carrageenan, alginate, guar gum, locust bean gum, psyllium, xanthan gum, gum arabic, fructo-oligosaccharides, inulin, and agar.

5. The supplement of claim 3, wherein the emulsifier is selected from the group consisting of propylene glycol monostearate, sodium stearoyl lactylate, calcium stearoyl lactylate, a monoglyceride, a diglyceride, a mono-diglyceride a polyglycerol ester, a lactic acid ester, polysorbate, a sucrose ester, a diacetyl tartaric acid ester of mono-diglycerides, and a citric acid ester of monoglycerides.

6. The supplement of claim 1, wherein the supplement comprises at least one vitamin.

7. The supplement of claim 6, wherein the at least one vitamin is one or more of beta-carotene, biotin, folic acid, niacin, pantothenic acid, riboflavin, thiamin, vitamin B₁₂, vitamin B₆, vitamin C, vitamin D₃, vitamin E, and vitamin K.

8. The supplement of claim 1, wherein the supplement comprises at least one mineral.

9. The supplement of claim 8, wherein the at least one mineral is one or more of calcium, chromium, copper, iron, magnesium, manganese, potassium, selenium, and zinc.

10. The supplement of claim 1, wherein the supplement is in the form of a food product.

11. The supplement of claim 10, wherein the food product is a food bar.

12. The supplement of claim 10, wherein the food product is a semi-solid.

13. The supplement of claim 10, wherein the food product is a beverage.

14. A method of treating iron deficiency in an individual, the method comprising administering to the individual an effective amount of the supplement of claim 1.

15. The method of claim 14, wherein the individual is a female human of child-bearing age.

16. The method of claim 14, wherein the individual is a child or an adolescent.

17. The method of claim 14, wherein the individual is a kidney dialysis patient.
